(19) United States
(12) Patent Application Publication
(10) Pub. No.: US 2010/0210930 A1
(43) Pub. Date: Aug. 19, 2010

(54) Physiological Blood Gas Detection Apparatus and Method

(76) Inventor: Stephen D. Saylor, South Hamilton, MA (US)

Correspondence Address: PEPPER HAMILTON LLP, ONE MELLON CENTER, 50TH FLOOR, 500 GRANT STREET, PITTSBURGH, PA, 15219, US

(21) Appl. No.: 12/706,479

(22) Filed: Feb. 16, 2010

Related U.S. Application Data
(60) Provisional application No. 61/152,489, filed on Feb. 13, 2009.

(51) Int. Cl. A61B 5/00

(52) U.S. Cl. 600/323

(57) ABSTRACT

The present disclosure is directed to methods and physiological sensing devices for determining blood gas concentrations in a human or animal subject. Such devices may further detect and measure the subject's heart rate.

### RELATED APPLICATIONS

This application claims the benefit and priority of provisional patent application Ser. No. 61/152,489 filed on Feb. 13, 2009, all of which is hereby incorporated by reference.

### TECHNICAL FIELD

The present disclosure relates to devices and methods for non-invasively detecting physiological blood gas levels in a subject. More specifically, the present invention is focused on pulse and blood oxygen levels in a desired subject.

### BACKGROUND

Pulse oximeters are non-invasive devices for detecting, reading, and monitoring blood oxygen concentrations in a patient. Specifically, hemoglobin is the metalloprotein found in the red blood cells that transport oxygen. Both the oxygenated hemoglobin (HbO₂) and the deoxygenated hemoglobin (Hb) have unique light absorption properties as shown by the graph in FIG. 1. Notably, Hb and HbO₂ readily absorb light having a wavelength (λ) of ˜660 nm and ˜960 nm, respectively. FIG. 1 is a common graph plotting the light having wavelengths vs. molecular extinction coefficient (ε). Molecular extinction coefficient is a constant defined as the optical density of a sample of 1 mmol⁻¹, measured with a path length of one cm. Currently, oximeters are mainly comprised of a red LED emitting at ˜660 nm, an IR LED emitting at ˜910 nm and a standard photodiode. Most oximeters arrange the LEDs to be in optical contact with a patient's finger with a photodiode arranged on the opposite side of the finger. In this configuration, the red wavelengths and IR wavelengths are able to pass through the tissue of the finger to be absorbed by the HbO₂ and Hb respectively. Once the red and IR electromagnetic radiation have penetrated the finger and a portion is absorbed by the hemoglobin the remaining photons are detected, measured by the photodetector then calculated to obtain the R/IR ratio to determine the blood oxygen saturation (SpO₂).

Pulse oximeters have many shortcomings that adversely affect the accuracy of the monitored and calculated blood gas results. As a result, the present disclosure provides devices and methods that seek to improve the accuracy and overall perform of such and similar devices.

### SUMMARY

The following disclosure provides methods, and apparatuses for obtaining novel physiological sensing devices, in particular pulse oximeters. Embodiments hereof provide a method and device for determining blood gas concentrations in a human or animal subject.

In one embodiment of the present invention, a physiological sensing device can include at least one broadband spectral electromagnetic radiation emitter and at least one silicon based photosensing diode for detecting multiple wavelengths of electromagnetic radiation from the broadband radiation emitter. Notably, the photosensing diode may include an active area or region which has been treated by a laser, preferably a pulsed femtosecond laser to alter and enhance the absorption properties of the diode.

Implementations of the device may include one or more of the following features. The device may include a substrate layer and the substrate layer may be flexible. The device may further include a conformable housing to house the physiological sensing device. In some implementations, the at least one silicon based photosensing diode is a single diode. In other implementations, the at least one silicon based photosensing diode may include at least one textured surface. The at least one textured surface may be formed by a laser process. The device may include a feature wherein the photosensing diode can detect electromagnetic radiation having at least one wavelength in the range of about 200 nm to about 2,500 nm. In other implementations, the device may include a feature wherein the photosensing diode can detect electromagnetic radiation having at least one wavelength in the range of about 600 nm to about 1,300 nm. The device may include a feature wherein the substrate layer has a thickness of less than about 0.5 mm. In some implementations, the at least one silicon based photosensing diode is partially flexible.

The physiological sensing device may include the feature of being capable of detecting and measuring oxygen saturation in the blood of a human or animal. The physiological sensing device may be configured to detect or measure at least one physiological element from the group consisting of blood glucose levels, carbon monoxide levels, carbon dioxide levels and methemogloblin levels in a human or animal subject. In some implementations the silicon based photosensing diode may have an external quantum efficiency of greater than 100%. The device may further comprise a computer means for calculating the oxygen saturation in the blood (SpO₂). The device may further comprise at least one filter disposed over said photosensing diode. The device may have the feature wherein the silicon based photosensing diode has a responsivity greater than about 0.8 amps/Watt for a range of wavelengths of incident electromagnetic radiation greater than about 1050 nm.

In general, in another embodiment of the present invention, a pulse oximeter device is disclosed. The pulse oximeter device includes at least one electromagnetic radiation emitter. The pulse oximeter device further includes a silicon based photosensing diode for detecting multiple wavelengths of electromagnetic radiation from the at least one electromagnetic emitter, wherein at least one wavelength is greater than 1050 nm, and the photosensing diode is operated at a bias less than about 30 volts.

Implementations of the device may include one or more of the following features. The device may include a feature wherein the photosensing diode is operated at a bias less than about 5 volts. The device may further include a feature wherein the photosensing diode has an external quantum efficiency greater than 30% for wavelengths greater than 1100 nm and having a thickness of less than 200 μm. In other implementations, the device may include a feature wherein the photosensing diode has a thickness of less than 100 μm and has an external quantum efficiency greater than 30% for wavelengths greater than 1100 nm. The device may include a feature wherein the photosensing diode has an external quantum efficiency greater than 50% for wavelengths greater than 1050 nm and has a thickness less than 200 μm. In other implementations, the device may include a feature wherein the photosensing diode has a thickness of less than 100 μm and has an external quantum efficiency greater than 50% for wavelengths greater than 1050 nm.

The present invention is also drawn towards methods for determining blood gas levels in a human or animal appendage exposed to electromagnetic radiation having two different wavelengths. Such methods may include the following steps: (a) generating electromagnetic radiation having first and second wavelengths; (b) exposing the selected appendage to the first and second electromagnetic radiations; (c) detecting the first and second electromagnetic radiations passing through the appendage with a silicon based photosensing diode; and (d) calculating the pulse and oxygen saturation levels in the human or animal from the detected radiation.

Implementations of the method may include one or more of the following features. The blood gas levels may be selected from a group consisting of oxygen saturation, carbon monoxide, carbon dioxide, blood glucose and methemogloblin levels. The method may further include the feature wherein the first or second wavelength is greater than 1150 nm.

Other uses for the methods and apparatus given herein can be appreciated by those skilled in the art upon comprehending the present disclosure.

### BRIEF DESCRIPTION OF THE DRAWINGS

For a fuller understanding of the nature and advantages of the present invention, reference is being made to the following detailed description of preferred embodiments and in connection with the accompanying drawings, in which:

FIG. 1 is a graphical representation of optical absorption properties of oxygenated and deoxygenated hemoglobin.

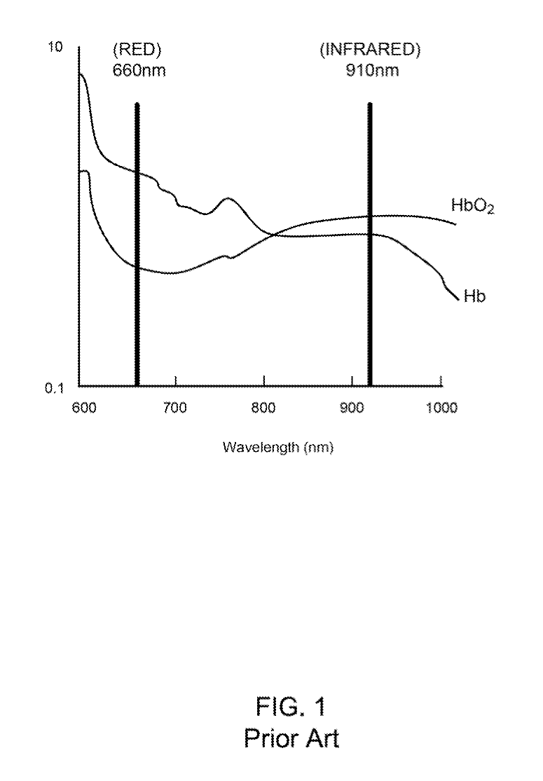

FIG. 2 is a graphical representation of optical absorption properties of oxygenated and deoxygenated hemoglobin according to some embodiments hereof;

FIG. 3 illustrates a physiological sensing device detecting electromagnetic radiation with a single photosensing diode according to an embodiment;

FIG. 4 illustrates a physiological sensing device detecting electromagnetic radiation with two photosensing diodes according to another embodiment; and

FIG. 5 illustrates a physiological sensing device detecting electromagnetic radiation with a silicon photosensing diode and a textured silicon photosensing diode according to another embodiment.

FIG. 6 illustrates a physiological sensing device including a light emitter, photosensing diode and an optical filter.

FIG. 7 illustrates a physiological sensing device detecting electromagnetic radiation from ambient light with a single silicon based photosensing diode according to an embodiment.

### DETAILED DESCRIPTION

As alluded to above, the present disclosure describes devices and method measuring and monitoring physiological conditions (i.e. blood gas concentrations) in a human or animal subject.

A physiological sensing device capable of detecting, measuring and calculating blood oxygen saturation, blood glucose levels, carbon monoxide levels, carbon dioxide levels and methemogloblin levels of a human or animal subject may be provided. In addition to detecting blood gas concentrations the physiological device may be capable of detecting the heart rate or pulse of a human subject.

As mentioned above, conventional pulse oximeters detect and measure the absorption of two wavelengths and determine the blood oxygen saturation SpO₂ in a human. This is of course assuming that there are only two species of hemoglobin present in the subject's blood. The equation (1) below defines the calculation that is performed by the oximeter, where SpO₂ is the measured blood oxygen saturation, HbO₂ and Hb represent oxygenated hemoglobin and deoxygenated hemoglobin, respectively.

SpO₂=HbO2/(Hb+HbO₂) (1)

However, there are many limitations with the current pulse oximeters. Some limitations are, motion, low perfusion, venous pulsations, venous congestion, light interference, optical noise interference, intravascular dyes, fingernail polish, Low SpO₂ (less than 70%), sensor site temperature, tissue malformations, tissue scars and burns, to name a few. The methods and apparatus disclosed herein attempt to overcome some of these limitations.

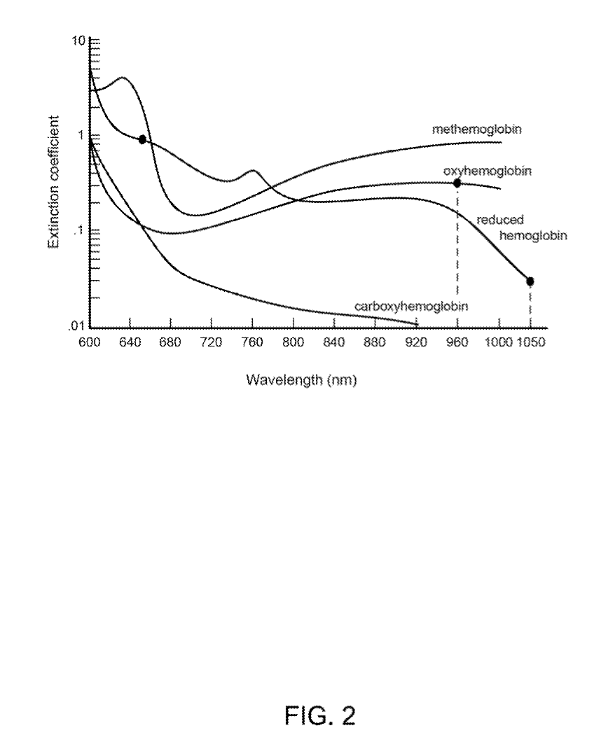

Referring to FIG. 2, the light absorption properties of oxygenated hemoglobin (HbO₂) and the deoxygenated hemoglobin (Hb) are shown for longer wavelengths. Through the use of an electromagnetic radiation emitter that is capable of emitting the longer wavelengths shown in FIG. 2, along with an appropriate photosensing diode configured to detect longer wavelengths, many of the limitations of current pulse oximeters can be overcome. Longer wavelengths of light are able to penetrate the patient's finger better with less interference.

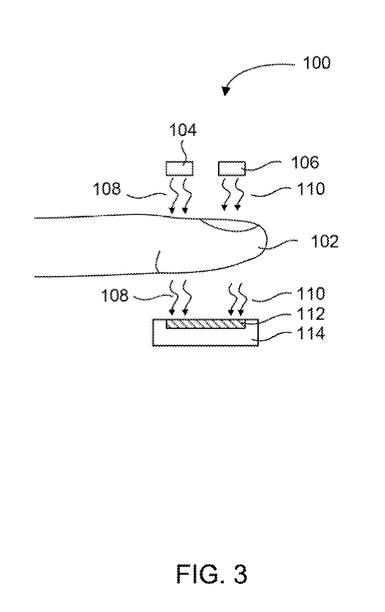

In one embodiment, a physiological sensing device may be provided having at least one electromagnetic radiation emitter and at least one photosensing diode for detecting multiple wavelengths of electromagnetic radiation from the at least one electromagnetic emitter. The photosensing diode can have an active area which has a portion that has been processed or treated such that the portion has a textured surface. The textured surface may be achieved through a laser process, through an etching process, the addition of material (i.e. quantum dots) or through any other known methods. FIG. 3 illustrates one embodiment as described above.

In FIG. 3 a physiological sensing device 100 is shown. The device may include one or more electromagnetic radiation emitters 104, 106 and at least one silicon based photosensing diode 114. The electromagnetic radiation emitter can be any light source that can emit light 108, 110 having the desired frequency and wavelengths. For example, the electromagnetic radiation emitter can be an incandescent light source or more preferably a light emitting diode (LED). In one embodiment the electromagnetic radiation emitter can be a single LED having a wavelength less than about 800 nm. More specifically, the emitter can be a red LED configured to emit light having a wavelength of about 660 nm. In some embodiments, a single electromagnetic radiation emitter such as an LED can be configured to emit at least one wavelength in the range of 200 nm to 2,500 nm. In another embodiment, the radiation absorbed by the human appendage 102 may originate from two LEDs 104 and 106. The first LED 104 can be configured to emit light having a wavelength less than about 800 nm and the second LED 106 can be configured to emit light having a wavelength of greater than about 800 nm. It is preferred to use wavelengths where maximum absorption can be achieved. It is also desired to pick two wavelengths that have maximum absorption and difference in wavelengths (Δλ). The difference in wavelengths can have a difference as little as 20 nm or as much as 1000 nm. In other embodiments it may be necessary to have at least two emitters with wavelengths differing more than 1000 nm. It mostly depends on the types of physiological properties being detected and measured. One skilled in the art will appreciate that more than two LEDs may be used with the present invention to achieve different or better results. For example, using wavelengths higher in the infrared region may allow for better skin and tissue penetration, regardless of the tissue thickness, or whether the subject appendage contains burnt and/or scar tissue. In other embodiments, ambient light can be substituted for the LED or incandescent light sources.

In yet another embodiment of the present invention, two electromagnetic radiation emitters 104 and 106 capable of emitting light 108 and 110 having wavelengths of about 660 nm and 1050 nm are used. Once the light is emitted it can pass through hair, nail, tissue and bone of the subject's appendage 102. A portion of the red and infrared light is absorbed by the Hb and HbO₂. The unabsorbed portion or light intensity of the two wavelengths can be measured by the silicon based photosensing diode 114. Further, the photosensing diode 114 may include a textured surface portion 112 located near the surface of the diode. The textured surface portion 112 may be formed through a laser treatment or alternatively through other texturing process known to those skilled in the art. The textured surface portion 112, helps improve broadband light detection and sensitivity which allows the silicon based photosensing diode 114 to be operated at a bias of less than 30 volts while effectively detecting at least one wavelength 1050 nm or greater. In alternate embodiments, the textured surface portion 112, allows the silicon based photosensing diode 114 to detect at least one wavelength of 1050 nm or greater while the photosensing diode 114 is being operated at a bias of less than 5 volts. In the preferred embodiment, the silicon based photosensing diode 114 is configured and operated in a reverse bias voltage configuration.

In some embodiments, a lower power LED light source can be used due to the higher penetration rate of the longer wavelength radiation that can be detected by the silicon based photosensing diode 114 which includes the textured surface portion 112. The textured surface portion 112 of the photosensing diode 114 may also allow for the elimination of the light emitting source in configurations that utilize ambient light for measurements. The photosensing diode 114 including the textured surface portion 112, may be constructed from silicon, which is lower cost than typical photosensors in current blood gas detection devices. The photosensing diode 114 including the textured surface portion 112, may also eliminate the need for copper shielding used in current typical photosensors in blood gas detection devices. Traditional blood gas detection devices suffer from too much measurement noise in relation to the photosensor signal. As a consequence, traditional devices are required to include copper shielding around the photosensing diode to reduce noise. Due to the improved broadband sensitivity the of the presently disclosed photosensing diode 114 the signal to noise ratio can be improved thereby allowing for, the copper shielding may be reduced or eliminated from the device. The physiological sensing device can include a computing means that can utilize algorithms for calculating the SpO₂. Alternatively, the emitters 104 and 106 may be oriented such that a portion of the emitted radiation can be absorbed by hemoglobin and reflected to the photosensing diode(s). In addition, the photosensing diode(s) may be located proximal the radiation emitters (not shown).

In most of the embodiments, the photosensing diode 114 contains a textured surface 112 that is a laser-treated or processed region. The textured surface can be on the top side (near incident light) or the bottom side or both. In an alternative embodiment, a non-bulk semiconductor material may be textured and disposed on or near the photodiode. The laser-treated region can improve the photo sensitivity of the device, enabling it to detect light having wavelengths from 200 nm-30 μm. This technology was developed and patented by Eric Mazur and James Carey, which can be found in U.S. Pat. Nos. 7,390,689; 7,057,256; 7,354,792; 7,442,629 which are incorporated by reference in their entirety. This technology has been coined the term of “Black Silicon.”

In an exemplary embodiment, a textured surface portion 112 of a photosensing diode 114 may be formed, for example, with femtosecond laser pulses, as disclosed in U.S. Pat. No. 7,057,256. The semiconductor material may include, without limitation, a doped semiconductor material, such as sulfur-doped silicon. In alternate embodiments, the textured surface portion 112 may be formed through an etching or similar process.

The photosensing diode 114 including the textured surface 112 may have a broad spectral response. In an embodiment, the diode may exhibit a photoelectric response to electromagnetic radiation in the visible and near infrared ranges. In an embodiment, the diode may have a photoelectric response to at least one wavelength, but not necessarily all, of light from about 250 nm to about 3500 nm. In another embodiment, the diode may have a photoelectric response to at least one wavelength, but not necessarily all, of light from about 250 nm to about 1200 nm. In yet another embodiment, the diode may have a photoelectric response to at least one, but not necessarily all, wavelengths of light from about 400 nm to about 1200 nm. Other wavelength ranges of photoelectric response in the visible and near infrared spectral ranges are encompassed within the scope of this disclosure. Specifically, the silicon based photosensing diode 114, with the textured surface portion 112 can detect at least one wavelength above 1050 nm while being operated at a bias of less than 30 volts. As described above, various embodiments of the invention including the silicon based photosensing diode 114, with the textured surface portion 112 can detect at least one wavelength above 1100 nm, 1200 nm, and 1300 nm while being operated at a bias of less than 30 volts.

In an exemplary embodiment, the photosensing diode 114 may exhibit a responsivity of greater than about 0.8 A/W for incident electromagnetic radiation having a wavelength greater than 1050 nm. The photosensing diode 114 including the textured surface portion 112 may have unique properties that allow for the diode to obtain an external quantum efficiency of greater than 100%. In another embodiment, the photosensing diode 114 including the textured surface portion 112 has total thickness of less than 100 μm and has an external quantum efficiency greater than 30% for at least one wavelength greater than 1100 nm. In addition, the same photosensing diode 114 can have an external quantum efficiency greater than 50% for at least one wavelength greater than 1050 nm and may exhibit a responsivity of greater than about 0.1 A/W for incident electromagnetic radiation having a wavelength greater than 1050 nm.

The photosensing diodes may include a base layer having a thickness of less than about 500 μm (not shown). The base layer may be incorporated into the photosensing diode during epitaxial growth. In some embodiments the base layer may be a back side contact for the diode. It may be comprised of a metal or metal alloy, for example, tin, tungsten, copper, gold, silver, aluminum or combinations or composites thereof Other metals and/or materials maybe used as the base layer that exhibit ohmic properties. Moreover, the base layer may be at least a partially flexible layer having the ability to bend and conform in any direction desired. In other embodiments the thickness of the base layer and photosensing diode is thin enough to allow the diode to also flex, partially flex or bend with the base layer.

In other aspects, a support substrate layer may be in contact with the electromagnetic radiation emitters and photosensing diodes. The substrate may be the housing for the emitter and diodes or it may be part of a housing device. Further, the substrate can have a thickness of less than about 0.5 mm. In this embodiment the substrate layer may be at least partially flexible or bendable. In this case, the substrate layer may have an attaching means (buckle, Velcro, clasp, or adhesive) for attaching to the desired appendage. In this embodiment the flexible substrate may bend around and affix to the finger like a bandage, thereby eliminating movement issues and device contact issues. In another aspect, a conformable housing may be used to house the radiation emitter and photodiode. The house may include foam, a sponge or other material that is able to provide comfort and allow the housing to mold about the appendage.

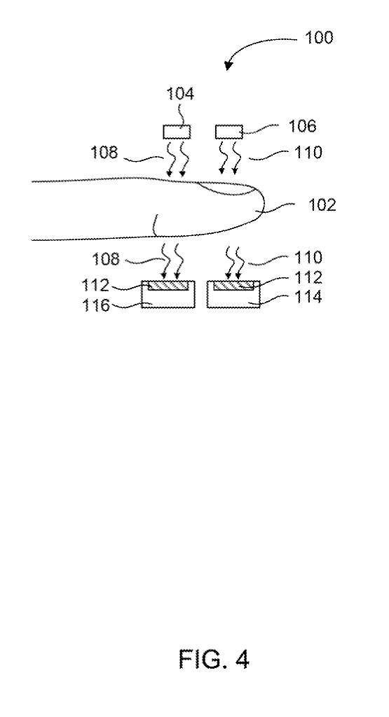

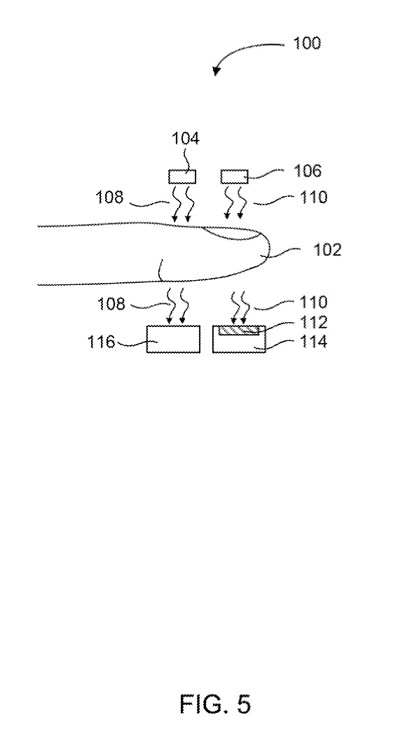

FIG. 4 depicts an alternative embodiment that may include a first and second electromagnetic radiation emitter, 104, 106 a first and second light having different wavelengths, 108, 110 an appendage 102, (i.e. finger or earlobe for receiving transmitted light) and a first and second photosensing diode 114, 116 being configured to receive light 108, 110 that has passed through the appendage 102. As noted above, the first and second photosensing diodes may include a textured surface region 112 located on or near the surface the photosensing diode. The first light 108 may have wavelength in the range of about 400 nm-800 nm and the second light 110 may have a wavelength in the range of about 800 nm-2,500 nm. Alternatively, the first photosensing diode 116 may be a conventional silicon photodiode devoid of a textured surface region capable of detecting light having a wavelength less than 800 nm, as shown in FIG. 5. In many of the embodiments disclosed herein, the photosensing diode can detect a portion of electromagnetic radiation in the range of about 400 nm-2,500 nm.

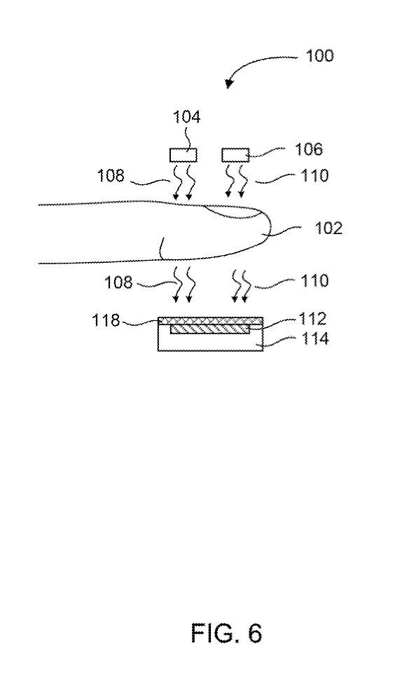

FIG. 6 illustrates another embodiment of a physiological sensing device 100, that may include electromagnetic radiation emitters 104, 106 for emitting desired light 108, 110 into an appendage 102. In addition, a photosensing diode 114 may be oriented in an optical path about the appendage to receive the emitted radiation. The photosensing diode 114 may further include a textured surface region 112 and at least one filter 118. The filter 118 is typically oriented or disposed above the active area or region of the photosensing diode 114. The filter can be any known color or light filter on the market that is capable of blocking or filtering out an undesired light. The filtering may improve or enhance the detection and measuring accuracy of the device.

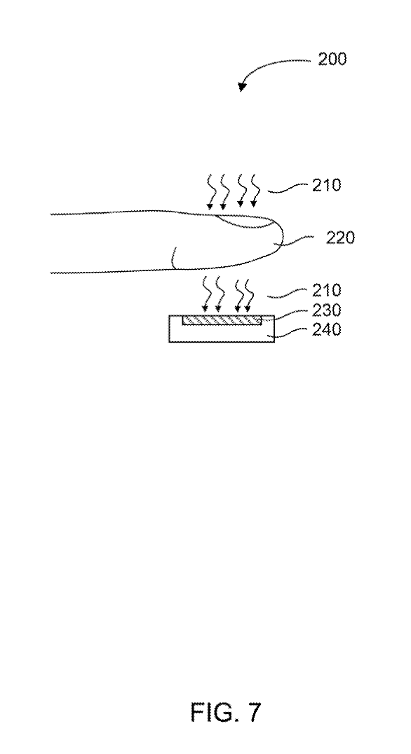

Referring to FIG. 7, a physiological sensing device 200 is configured to operate with ambient light 210. The ambient light 210 travels through the appendage 220. A silicon based photosensing diode 240 may be oriented in an optical path about the appendage to receive the ambient light 210 that has traveled through the appendage 220. The silicon based photosensing diode 240 may include a textured surface region 230.

Methods for determining blood gas levels in a human or animal appendage exposed to electromagnetic radiation having two different wavelengths may be provided. Such methods may comprise one or more of the following steps: (a) generating electromagnetic radiation having first and second wavelengths; (b) exposing the selected appendage to the first and second electromagnetic radiations; (c) detecting the first and second electromagnetic radiations passing through the appendage with at least one photodetector as described herein; and (d) calculating the pulse and oxygen saturation levels in a human or animal from detected radiation. Some of the blood gas levels determined may include but not limited to oxygen saturation, carbon monoxide, carbon dioxide, blood glucose and methemogloblin levels. While detecting the blood gas levels, the physiological device may further detect and calculate the human or animal subject's heart rate.

The present invention should not be considered limited to the particular embodiments described above, but rather should be understood to cover all aspects of the invention as fairly set out in the attached claims. Various modifications, equivalent processes, as well as numerous structures to which the present invention may be applicable, will be readily apparent to those skilled in the art to which the present invention is directed upon review of the present disclosure.

1. A physiological sensing device, comprising:
at least one electromagnetic radiation emitter; and
at least one silicon based photosensing diode for detecting multiple wavelengths of electromagnetic radiation from the at least one electromagnetic emitter.

2. The device of claim 1, further comprising a substrate layer.

3. The device of claim 2, wherein the substrate layer is flexible.

4. The device of claim 1, further comprising a conformable housing to house the physiological sensing device.

5. The device of claim 1, wherein the at least one silicon based photosensing diode is a single diode.

6. The device of claim 1, wherein the at least one silicon based photosensing diode includes at least one textured surface.

7. The device of claim 6, wherein the at least one textured surface is formed by a laser process.

8. The device of claim 1, wherein the photosensing diode can detect electromagnetic radiation having at least one wavelength in the range of about 200 nm to about 2,500 nm.

9. The device of claim 1, wherein the photosensing diode can detect electromagnetic radiation having at least one wavelength in the range of about 600 nm to about 1,300 nm.

10. The device of claim 2, wherein the substrate layer has a thickness of less than about 0.5 mm.

11. The device of claim 1, wherein the at least one silicon based photosensing diode is partially flexible.

12. The device of claim 1, wherein the physiological sensing device is configured to detect or measure at least one physiological element from the group consisting of blood glucose levels, carbon monoxide levels, carbon dioxide levels and methemogloblin levels in a human or animal subject.

13. The device of claim 1, wherein the silicon based photosensing diode has an external quantum efficiency of greater than 100%.

14. The device of claim 1, further comprising a computer means for calculating the oxygen saturation in the blood (SpO₂).

15. The device of claim 1, further comprises at least one filter disposed over said photosensing diode.

16. The device of claim 1, wherein the silicon based photosensing diode has a responsivity greater than about 0.8 amps/Watt for a first range of wavelengths of incident electromagnetic radiation.

17. A pulse oximeter device, comprising:
at least one electromagnetic radiation emitter; and
a silicon based photosensing diode for detecting multiple wavelengths of electromagnetic radiation from the at least one electromagnetic emitter; wherein at least one wavelength is greater than 1050 nm, wherein the photosensing diode is operated at a bias less than about 30 volts.

18. The device of claim 17, wherein the photosensing diode is operated at a bias less than about 5 volts.

19. The device of claim 17, wherein the photosensing diode has an external quantum efficiency greater than 30% for wavelengths greater than 1100 nm and having a thickness of less than 200 μm.

20. The device of claim 19, wherein the photosensing diode has a thickness of less than 100 μm.

21. The device of claim 17, wherein the photosensing diode has an external quantum efficiency greater than 50% for wavelengths greater than 1050 nm and has a thickness less than 200 μm.

22. The device of claim 21, wherein the photosensing diode has a thickness of less than 100 μm.

23. A method for determining blood gas levels in a human or animal appendage exposed to electromagnetic radiation having two different wavelengths, comprising the steps of
(a) generating electromagnetic radiation having first and second wavelengths;
(b) exposing the selected appendage to the first and second electromagnetic radiations;
(c) detecting the first and second electromagnetic radiations passing through the appendage with a silicon based photosensing diode; and
(d) calculating the pulse and oxygen saturation levels in the human or animal from detected radiation.

24. The method of claim 23, wherein the blood gas levels are selected from a group consisting of oxygen saturation, carbon monoxide, carbon dioxide, blood glucose and methemogloblin levels.

25. The method of claim 23, wherein the first or second wavelength is greater than 1150 nm.
